(19) United States
(12) Patent Application Publication
(10) Pub. No.: US 2024/0207857 A1
(43) Pub. Date: Jun. 27, 2024

(54) DROPLET PCR

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 18/557,285

(22) PCT Filed: Apr. 29, 2021
(86) PCT No.: PCT/US2021/029887

§ 371 Date: Oct. 25, 2023

(51) Int. Cl. B01L 7/00; B01L 3/02

(52) U.S. Cl. CPC B01L 7/52; B01L 3/0241; B01L 2200/0673; B01L 2200/16; B01L 2300/1805; B01L 2400/0487

(57) ABSTRACT

A droplet PCR system is described herein. The droplet PCR system comprises a chamber having a heating surface, a droplet dispenser to dispense PCR-reagent-containing droplets into the chamber, a heater disposed onto the heating surface of the chamber to heat a layer of fluid adjacent the heating surface, and a controller to control the heater to produce a pulsed heat flux.

### BACKGROUND

Polymerase chain reaction (PCR) is a method enabling in vitro replication of DNA. It relies on sequence hybridization and enzyme-based amplification. The enzyme used is DNA polymerase. The term “chain reaction” in this context means that the products of previous cycles serve as starting materials for the next cycle and thus enables exponential replication by repeating several times a series of temperature changes. Each cycle results in a doubling of the number of target DNA molecules and in theory after n cycles, 2ⁿ copies can be produced. In practice, the amplification process reaches a plateau as PCR reagents are depleted, preventing further amplification.

### BRIEF DESCRIPTION OF THE DRAWINGS

FIG. 1 is a schematic diagram of an example of a droplet PCR system.

FIG. 2A and FIG. 2B are schematic diagrams focused on the chamber of example chambers usable in example droplet PCR systems.

FIG. 2C and FIG. 2D are schematic diagrams focused on the arrangement between the chamber and the heater usable in example droplet PCR systems.

FIG. 3 is a schematic diagram of an example of a droplet PCR system.

FIG. 4 is a schematic diagram of another example of a droplet PCR system.

FIG. 5 is a schematic diagram of a further example of a droplet PCR system.

FIG. 6 is a schematic diagram of yet another example of a droplet PCR system.

FIG. 7A to FIG. 7C are schematic diagrams of example confiner arrangements usable in example droplet PCR systems.

FIG. 8 is a schematic diagram of yet a further example of a droplet PCR system.

FIG. 9 is a flowchart of an example droplet PCR method.

FIG. 10 is a flowchart of an example droplet PCR method.

FIG. 11 is a flowchart of an example droplet PCR method.

FIG. 12 is a flowchart of an example droplet PCR method.

FIG. 13 is a flowchart of an example droplet PCR method.

FIG. 14 is a flowchart of an example droplet PCR method.

FIG. 15 is a flowchart of an example droplet PCR method.

FIG. 16 is a flowchart of an example droplet PCR method.

FIG. 17 is a schematic diagram of an example computer system.

### DETAILED DESCRIPTION

PCR makes it possible to rapidly amplify a small DNA sample so that it is large enough to be studied. PCR methods can rely on exposing DNA to reagents. The two example reagents are primers and DNA polymerase. Example primers are short single stranded DNA fragments complementary to a portion of a target DNA segment. Digital PCR (dPCR) is a PCR method where the sample is distributed over a plurality of partitions—e.g. microwells, chambers or droplets—and the reaction is carried out in each partition individually. The volume of each partition can be in the femtoliter range although it can be larger. Variances in the amplification efficiency are decreased, sensitivity to PCR inhibiting chemicals is lowered, detection limits are improved, and quantitation of targets is more accurate.

Droplet PCR is a type of dPCR method where each partition corresponds to a droplet. The volume of the droplets can be of the picoliter to nanoliter range. Droplets in droplet PCR are created when needed so that the number of partitions can be adjusted to meet the requirement of an application. Volume variation of droplets is low, for example few percent, thus, unlike microwells and chambers, it does not depend on manufacturing homogeneity over a large array of features.

Droplet PCR can make use of heating and cooling cycles. Cooling and heating times can be relatively long because the droplets are submerged in a carrier fluid: and when the droplets are heated or cooled, so is the carrier fluid.

Various example droplet PCR systems and methods are disclosed herein. In these droplet PCR systems the cooling and heating times are shortened. These droplet PCR systems and methods use pulse heating to heat part of the carrier fluid. Pulse heating is a heating where heat flux is modulated between a higher level of heat flux and a lower level of heat flux. The lower level of heat flux can be different from 0. In some examples, the heat flux can be modulated to have a periodic varying waveform. The periodic varying waveform can be built up from a plurality of sinusoidal waveforms through Fourier decomposition. In some examples, the heat flux can be modulated to be provided in waves of single burst, i.e. the heat flux magnitude starts from a baseline, increases and return to the baseline. The baseline can be different from zero. The duration of a burst can be from 0.2 μs to 2 ms. In some examples, the duration of a burst can be from 2 μs to 500 μs. In some examples, the duration of a burst can be from 20 μs to 200 μs. The use of pulse heating makes it possible to heat a layer of fluid where the droplets are located while maintaining the fluid around the heated layer at a temperature lower than the temperature at which the heated layer is heated. During cooling the fluid surrounding the heated layer acts as a heat sink and speeds the cooling. This can also enable a subsequent heating to start faster. Therefore, these droplet PCR systems and methods provide relatively fast digital nucleic acid testing. Nucleic acid testing can be used in disease diagnostics such as diagnosis of infectious diseases.

In some example droplet PCR systems and methods disclosed herein, the droplets are driven to a desired location. The driven droplets can be discarded, or taken to analysis.

In some example droplet PCR systems and methods disclosed herein, analysis can rapidly take place with the provision of a detector. Detection can take place either when the droplets are in motion or motionless. Detection consists in detecting signals from the droplets, for example signals emitted by the PCR products inside the droplets resulting from amplification of a target DNA segment. Therefore, these droplet PCR systems and methods can provide quantitative determination of a target DNA segment from the number of droplets for which a signal has been detected. For example, the signal can be emitted by a fluorescent reporter dye, which is first attached to a probe and emits a signal once it is released from the primer by a DNA polymerase. Since the volume of sample preparation is distributed over the dispensed droplets, and since the volume of each droplet is substantively smaller than the volume of sample preparation, it is possible to consider that in each droplet there is either one target DNA segment or none. PCR happens in the droplets containing the target DNA segment. PCR results in an increase in number of copies of the target DNA segment, and the copies are rendered capable of emitting a signal. Thus, counting the number of droplets for which a signal is detected amounts to counting the number of the target DNA segment in the sample preparation. These droplet PCR systems and methods also provide multiplexing of target DNA segment determination.

In some example droplet PCR systems and methods disclosed herein, the droplets can be driven to a desired location or the reagents within the droplets can be mixed.

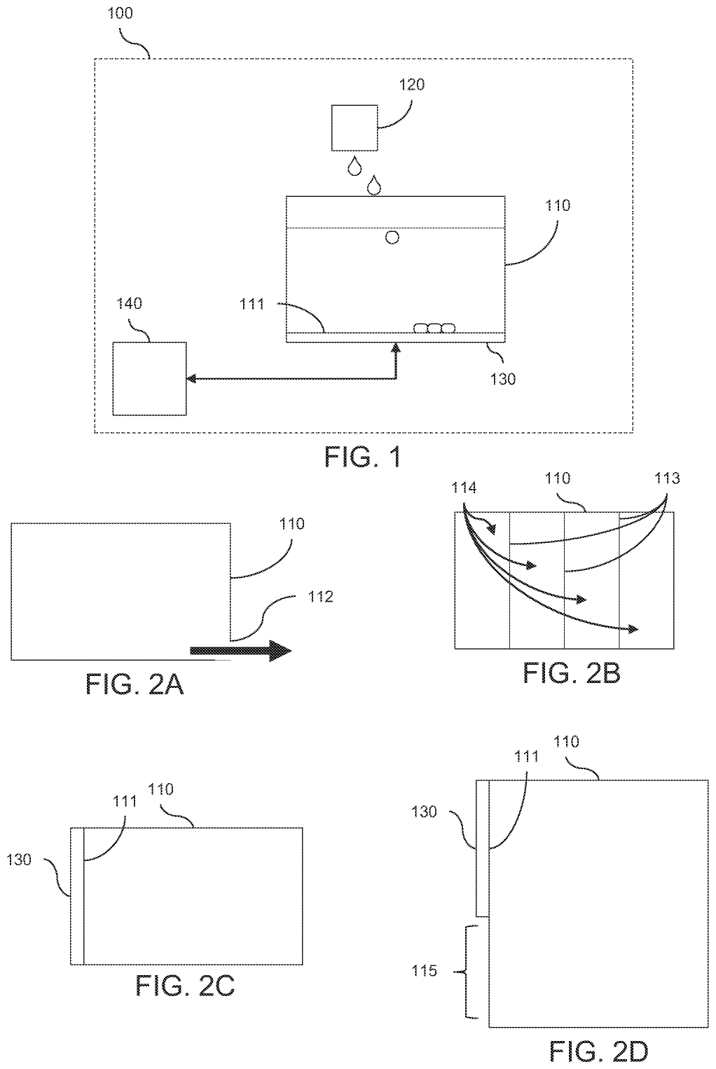

FIG. 1 is a schematic diagram of an example droplet PCR system 100. Droplet PCR system 100 comprises a chamber 110 having a heating surface 111, a droplet dispenser 120 to dispense PCR-reagent-containing droplets into the chamber 110, a heater 130 disposed onto the heating surface 111 of the chamber 110 to heat a layer of fluid adjacent the heating surface 111, and a controller 140 to control the heater to produce a pulsed heat flux. The droplet PCR system 100 is a system for performing PCR reactions in droplets. The diameter of the dispensed droplets can be of the micrometer range, such as 5 μm to 120 μm. In some examples, the diameter of the dispensed droplets can be from 7 to 60 μm. In some examples, the diameter of the dispensed droplets can be from 10 μm to 20 μm, for example 10 μm or 15 μm. The diameter of a droplet is the diameter of a sphere having a volume equal to the volume of the droplet. Therefore, in some examples, the volume of the droplet can be from 0.05 pL to 1 nL. In some examples, the volume of the droplet can be from 0.2 pL to 100 pL. In some examples, the volume of the droplet can be from 0.5 pL to 5 pL, for example 0.5 pL or 2 pL. The droplet diameter can be measured using a variety of methods such as taking an image of the droplet. When an image of a droplet is taken, the outline of the droplet can appear as a double outline. The midpoint of this double outline is measured as the diameter of the droplet. Another method is using Fraunhofer diffraction method where droplets are directed to the path of a laser beam, thus scattering the direction of the laser rays creating a diffraction pattern. The diffraction pattern depends on the size of the droplet.

The chamber 110 can be a partially enclosing vessel or recipient, i.e. it can comprise an opening receiving the dispensed droplets. The chamber 110 can comprise a bottom surface and one or a plurality of side surfaces. The terms “side” and “bottom” are to be construed with respect to the position of the chamber 110 in operation. Thus, the “bottom surface” can correspond to the lowest surface of the chamber 110, while a “side surface” can correspond to a surface which is capable of containing a liquid in a direction perpendicular to gravity. The surfaces of the chamber 110 can be made of various materials such as polystyrene, polypropylene, polycarbonate, cyclo-olefins, glass, and quartz. In some examples, the surfaces of the chamber 110 are made of polycarbonate.

A carrier fluid can be provided to the chamber 110 so that the chamber 110 can be filled with a carrier fluid. Thus, in some example droplet PCR systems, the droplet PCR system 100 can comprise a chamber 110 filled with a carrier fluid and having a heating surface 111: a droplet dispenser 120 to dispense PCR-reagent-containing droplets into the chamber 110; a heater 130 disposed onto the heating surface 111 of the chamber 110 to heat a layer of fluid adjacent the heating surface 111: and a controller 140 to control the heater 130 to produce a pulsed heat flux.

A carrier fluid is a fluid which is immiscible or hardly miscible with the droplets. In operation, the carrier fluid forms a continuous phase, surrounds the droplets and prevents two or more droplets from merging together to form a single daughter droplet. In some examples, the droplet dispenser 120 can dispense aqueous droplets and the carrier fluid can be an oil. By “aqueous”, it is understood that the droplets comprise water as solvent in which the other compounds are dissolved. An oil is a nonpolar viscous liquid which is hydrophobic. The oil can be an organic oil or a mineral oil. An example of organic oil can be a vegetable oil such as sunflower oil, i.e. comprising, for example, linoleic acid and oleic acid. An example of mineral oil can be petroleum distillates, such as an oil comprising saturated naphthenic hydrocarbons or saturated paraffinic hydrocarbons. The oil can be a silicone oil such as linear polydimethylsiloxane. The oil can comprise an alkane which is liquid at room temperature, such as hexadecane. The oil can be a perfluorinated oil. The carrier fluid can comprise a surfactant to decrease the likelihood of droplets merging together. The surfactant is a compound that lowers the surface tension between the carrier fluid and the liquid of the droplets and stabilizes the interface between the carrier fluid and the droplets. The surfactant can also prevent some PCR reagents such as DNA polymerase from concentrating at the interface between the carrier fluid and the droplets. The surfactant can be a silicon-based surfactant.

The chamber 110 can have a dimension taken perpendicularly to a portion of the heating surface 111 of at least 50 μm. In some examples, this dimension can be at least: 100 μm, 200 μm, 500 μm, and 1 mm.

The heating surface 111 is a surface of the chamber 110 through which the droplets are heated. The heating surface 111 can be a side surface or a bottom surface. In some examples, the heating surface 111 can be a portion or the entirety of the bottom surface of the chamber 110. In some examples, the heating surface 111 can be a portion or the entirety of the side surface of the chamber 110. In some examples, the heating surface 111 can be flat. In some examples, the heating surface 111 can be conical. In some example, the heating surface 111 can be frustoconical. In some example, the heating surface 111 can present some roughness and any orientation with respect to the surface, such as a perpendicular direction, will be taken from a mean plane averaging the level of the micro bumps.

The droplet dispenser 120 is a device capable of forming droplets. The droplet dispenser 120 can be a thermal ejector also called “inkjet nozzle”, a piezo-inkjet nozzle, an acoustic droplet ejector, a microfluidic droplet generator, an electrospinner, and a nanodispensing device. In some examples, the droplet dispenser 120 can be a thermal ejector, where the thermal ejector can heat fluid in a cavity proximate the thermal ejector to cause formation of a bubble in such fluid. Formation of the bubble in turn causes displacement of fluid proximate an orifice of the cavity. Displacement of the fluid can cause ejection of some of the fluid in the form of at least one fluidic droplet. Ejection of fluid by a thermal ejector can be referred to as thermal ejection and/or thermal jetting. The droplet dispenser 120 can comprise a plurality of thermal ejectors, piezo-inkjet nozzles, acoustic droplet ejectors, microfluidic droplet generators, electrospinners, and nanodispensing devices, to dispense a plurality of droplets at the same time. For example, in some examples, the droplet dispenser 120 can comprise a plurality of thermal ejectors.

A microfluidic droplet generator is a device having a microfluidic channel network in which two immiscible fluids are injected. The flow of one of the fluids is changed into droplets while the other fluid acts as a carrier fluid. There are passive microfluidic droplet generators and active microfluidic droplet generators. Passive microfluidic droplet generators are droplet generators that generate droplets without provision of external energy, such as co-flowing passive microfluidic droplet generators, cross-flowing passive microfluidic droplet generators and flow focusing passive microfluidic droplet generators. Active microfluidic droplet generators are droplet generators that generate droplets using additional energy input—such as electric, magnetic, and centrifugal energy—in promoting interfacial instabilities for droplet generation, such as those using a pneumatic chopper based on a polydimethylsiloxane valve design to physically break up the fluid stream, pulsed electric fields across a contraction to draw the aqueous phase in and break it off, and electro-wetting to adjust the wettability of the two phases. Droplets can also be dispensed from a microporous membrane through which the sample preparation is pushed.

The droplet dispenser 120 can be connected to a sample reservoir containing sample preparation to dispense to provide each droplet with the sample preparation. The sample preparation can be a solution of DNA or RNA in a reaction buffer. The DNA or RNA can be previously extracted from a sampled tissue of interest from an animal or a plant, a sampled fluid from an animal or a plant, or a microorganism such as a bacterium or a virus. The DNA can be a full chromosome, a plurality of full chromosomes, or fragments of one or more chromosomes. DNA can be single-stranded DNA or double-stranded DNA. In some examples, DNA can be double-stranded DNA. RNA can be identical RNA fragments or different RNA fragments. The sample preparation can comprise DNA alone in the reaction buffer or in a mixture with nucleotides, DNA polymerase, or a mixture thereof in the reaction buffer. The DNA polymerase is an enzyme which binds to a DNA strand and a primer and catalyzes the synthesis of a new complementary DNA strand from nucleotides.

A primer is a single stranded DNA that serves as a starting point for DNA replication by the DNA polymerase. It is composed of nucleobases, for example from 18 to 24 nucleobases, although it can be shorter or longer. It codes for a specific site from which replication is desired. Primers are used in pairs. Each pair of primers defines a target DNA segment and induces DNA replication towards each other which leads to the selected amplification of the target DNA segment between them if the target DNA segment is initially present in the droplet.

The nucleotides are DNA building blocks that the DNA polymerase transforms into nucleobases during replication to form a single-stranded DNA.

The sample preparation can comprise RNA alone in a reaction buffer or in a mixture with nucleotides, reverse transcriptase, DNA polymerase, or a mixture thereof in a reaction buffer. The reverse transcriptase is an enzyme which binds to an RNA to generate complementary DNA from RNA. The generated DNA is then amplified by the DNA polymerase.

The sample preparation can comprise DNA intercalating dyes. DNA intercalating dyes are dyes that intercalate into double-stranded DNA. Upon interaction with double-stranded DNA, the DNA intercalating dyes are stabilized into an excited state that and emit fluorescence. When the target DNA segment defined by the used primers is present in a droplet, there is amplification of the target DNA segment leading to an increase in amount of double-stranded DNA and a detectable level of fluorescence.

The droplet dispenser 120 can be connected to primer set reservoirs, each reservoir comprising a different primer set, each primer set being composed of a plurality of different primers. The droplet dispenser 120 can provide each droplet with a desired primer set to amplify the corresponding target DNA segment or segments by drawing primers from selected primer set reservoir or reservoirs. The number of primer set reservoirs can be small to very large. In some examples, the number of primer set reservoirs is from 1 to 50,000, in some examples from 1,000 to 25,000, and in some examples from 5,000 to 10,000. Each primer set can comprise a pair of primers to amplify a unique target DNA segment. Each primer set can comprise a plurality of pairs of primers to amplify a plurality of target DNA segments between each pair of primers. Each primer set can comprise degenerate primers, i.e. primers that are similar to each other but still different to amplify one particular gene of interest, because different codons—succession of three nucleobases—can code for the same amino acid. The droplet dispenser 120 can dispense droplets, each droplets being provided with a particular mixture of primers. The mixture of primers can result from the droplet dispenser 120 drawing primers from one or more primer set reservoirs. In some examples, a plurality of droplets can be provided with the same mixture of primers. In some examples each droplets can be provided with a different mixture of primers.

The primers can be free floating primers or attached to particles, e.g. magnetic particles, to allow solid phase amplification.

Each primer set can comprise corresponding fluorescent-labelled oligonucleotide probes. Fluorescent-labelled oligonucleotide probes corresponding to a primer set should be construed as fluorescent-labelled oligonucleotide probes designed to hybridize with the same target DNA segments as the primers but in different locations. Fluorescent-labelled oligonucleotide probes are single-stranded DNA comprising a fluorescent reporter dye located at their 5′ end. A fluorescent reporter dye is a component attachable to an oligonucleotide and which emits fluorescence when detached or released from the oligonucleotide. Different fluorescent reporter dyes emitting fluorescence signals at different wavelengths can be used to detect different target DNA segments within a single droplet. In operation, fluorescent-labelled oligonucleotide probes hybridize to the target DNA segments and are cleaved by the activity of the DNA polymerase leading to release of the fluorescent reporter dye and emission of fluorescence signals. The fluorescent-labelled oligonucleotide probes can be provided in the sample preparation. Other types of probes can be used such luminescent-labelled oligonucleotide probes or radioactively labelled oligonucleotide probes.

The droplet dispenser 120 can be connected to a DNA polymerase reservoir to provide each dispensed droplet with DNA polymerase. The dispenser 120 can be connected to a reverse transcriptase reservoir to provide each dispensed droplet with reverse transcriptase. The dispenser 120 can be connected to a nucleotide reservoir to provide each dispensed droplet with nucleotides. The dispenser 120 can be connected to a DNA intercalating dye reservoir to provide each dispensed droplet with DNA intercalating dyes.

The droplet dispenser 120 can be immobile to dispense droplets at a single location over the chamber 110. The droplet dispenser 120 can be movable with respect to the chamber 110 to dispense droplets at a plurality of locations over the chamber 110. The droplet dispenser 120 can be movable with respect to the chamber 110 along one direction to dispense droplets in a line pattern. The droplet dispenser 120 can be movable with respect to the chamber 110 along two directions to dispense droplets in a grid pattern. The droplet dispenser 120 being movable with respect to the chamber 110 means that the droplet dispenser 120 is movable and the chamber 110 is immobile, or the droplet dispenser 120 is immobile and the chamber 110 is movable, or both the droplet dispenser 120 and the chamber 110 are movable.

The heater 130 is disposed onto the heating surface 111 of the chamber 110. The heater 130 can be placed inside or outside the chamber 110. When the heater 130 is placed inside the chamber 110, one surface of the heater 130 is contacting a surface of the chamber 110. In some examples, the surface of the heater 130 opposite the one surface contacting the surface of the chamber 110 can be the heating surface 111.

In some examples, the heater comprises a metal layer. In some example the metal layer can be a standalone metal foil. The thickness of the metal foil can be 50 nm to 10 μm. In some examples, the thickness of the metal foil can be 100 nm to 5 μm. In some examples, the thickness of the metal foil can be 500 nm to 1 μm. The metal of the metal foil can be stainless steel, brass or aluminum.

In some examples, the metal layer can be a metal film deposited on the corresponding wall of the chamber or on a substrate. The metal film can be deposited using microfabrication technology onto the heating surface 111. The thickness of the metal film can be 1 μm to 100 μm. In some examples, the thickness of the metal film can be 5 μm to 50 μm. In some examples, the thickness of the metal film can be 10 μm to 25 μm. The metal film can be one of an indium tin oxide (ITO) layer, an aluminum-doped zinc oxide (AZO) layer, a chrome layer, an aluminum layer, a gold layer, a gold on chrome layer, a platinum layer, a tantalum layer and a nickel layer.

The heater 130 can have sections independently controlled to heat at different temperatures. In some examples where the heater 130 can be placed on a side surface of the chamber 110, the heater 130 can be divided into horizontal sections stacked in a vertical direction. Vertical and horizontal are to be construed with respect to gravity, the vertical direction being collinear with the gravitational force while the horizontal direction is perpendicular to the gravitational force.

The heater 130 can provide surface heat flux from 0.5 to 100 MW/m² pulsing, in some examples from 0.7 MW/m² to 0.9 MW/m², in some examples from 2 MW/m² to 3 MW/m², in some examples from 7 MW/m² to 9 MW/m², in some examples from 20 MW/m² to 30 MW/m², in some examples from 70 MW/m² to 90 MW/m².

The controller 140 controls the heater 130 to produce a pulsed heat flux causing heating of a layer of fluid adjacent the heating surface 111. The fluid adjacent the heating surface has a thickness along a direction normal to the heating surface, whereby the thickness of the heated layer of fluid can be in some examples up to twice the diameter of the droplets. The thickness of the heated layer of fluid can be smaller than the diameter of the droplets. For example, the thickness of the heated layer of fluid can be 0.5 μm to 20 μm, or 0.5 μm to 4 μm, or 10 μm to 20 μm.

In some examples, the controller 140 can control the heater 130 to provide a surface heat flux of 2 MW/m² to 3 MW/m² pulsing during 0.5 ms to 2 ms per cycle to heat a layer of fluid with thickness of 10 μm to 15 μm: for example a surface heat flux of 2.5 MW/m² during 1 ms per cycle to heat a thickness of 12 μm.

In some examples, the controller 140 can control the heater 130 to provide a surface heat flux of 7 MW/m² to 9 MW/m² pulsing during 0.05 ms to 0.2 ms per cycle to heat a layer of fluid with thickness of 3 μm to 5 μm: for example a surface heat flux of 8 MW/m² during 0.1 ms per cycle to heat a thickness of 4 μm.

In some examples, the controller 140 can control the heater 130 to provide a surface heat flux of 0.7 MW/m² to 0.9 MW/m² pulsing during 5 ms to 20 ms per cycle to heat a layer of fluid with thickness of 30 μm to 50 μm: for example a surface heat flux of 0.8 MW/m² during 10 ms per cycle to heat a thickness of 40 μm.

In some examples, the controller 140 can control the heater 130 to provide a surface heat flux of 20 MW/m² to 30 MW/m² pulsing during 0,005 ms to 0.02 ms per cycle to heat a layer of fluid with thickness of 1 μm to 1.5 μm: for example a surface heat flux of 25 MW/m² during 0.01 ms per cycle to heat a thickness of 1.2 μm.

In some examples, the controller 140 can control the heater 130 to provide a surface heat flux of 70 MW/m² to 90 MW/m² pulsing during 0.0005 ms to 0.002 ms per cycle to heat a layer of fluid with thickness of 0.3 μm to 0.5 μm: for example a surface heat flux of 80 MW/m² during 0.001 ms per cycle to heat a thickness of 0.4 μm.

In some examples where the heater 130 is divided into horizontal sections, the controller 140 can control the heater 130 so that adjacent sections heat the layer of fluid at different temperatures.

FIG. 2A is a schematic diagram focused on the chamber 110. In FIG. 2A, the chamber 110 can comprise an outlet 112 to discharge the droplets out of the chamber 110. The outlet 112 is a passage from the inside of the chamber 110 to the outside of the chamber 110. The chamber 110 of FIG. 2A can enable discharge of the droplets so that the chamber 110 can be used for a relatively longer continuous time. The chamber 110 of FIG. 2A can enable a continuous analysis of the droplets. The outlet 112 can be in this example positioned at the lowest part of the chamber 110. The outlet 112 can be connected to a connecting channel to fluidly connect the chamber 110 with another element. The outlet 112 can be a port. In such case, the port can be coupled with a connector at the end of a channel to fluidly connect the chamber 110 with another element. The port and the end of the channel can have complementary shapes to be fitted with one another. The outlet 112 can comprise a protrusion protruding outward from the chamber. In such case, the protrusion can be coupled with an end of a channel, for example by inserting the protrusion into the end of the channel.

In FIG. 2B, the chamber 110 can have one or a plurality of dividers 113. The one or a plurality of dividers 113 can form compartments 114 within the chamber 110. The divider 113 can contribute to divide the volume of the chamber 110 into smaller volumes and ease distinction of the dispensed droplets into groups of droplets. The droplets can thus be distributed over the compartments. Each compartment 114 can be dimensioned to receive a plurality of droplets on its floor formed by the heating surface 111. The divider 113 can be a wall extending from the bottom surface of the chamber 110. The chamber 110 can have a plurality of dividers 113. The wall can be flat or curved. The wall can be circular. The number of compartments 114 determined by the plurality of dividers 113 can be from few units to two million. In some examples, the number of compartments 114 is from 5 to 4,000, in some examples from 10,000 to 90,000, in some examples from 100,000 to 900,000, and in some examples from one million to 2 million.

Other examples of chamber 110 can be well trays and microwell trays: the wells being the compartments 114. In some examples, each well has a volume of few picoliters to few milliliters, in some examples a volume of 1 μL to 10 mL, and in some examples a volume of 20 pL to 500 nL.

The wells can be grouped into arrays with the same number of wells. The number of arrays per tray can be from 2 to 100.

The features of the example of FIG. 2A can be combined with the feature of FIG. 2B. In some examples, there can be an outlet per compartment to drive the droplets out of each compartment.

FIG. 2C is a schematic diagram focused on an example arrangement between the chamber 110 and the heater 130. While in the example represented in FIG. 1, the heating surface 111 is a bottom surface of the chamber 110 so that the heater 130 is placed against the bottom surface, in this example of FIG. 2C, the heating surface 111 can be a side surface of the chamber 110 and the heater 130 can be placed against the side surface. Also, while in FIG. 1 the heating surface 111 is illustrated as covering the whole bottom surface of the chamber 110, in this example of FIG. 2C the heating surface 111 is illustrated as covering the whole side surface of the chamber 110. Having the heater 130 against the side surface can enable taking advantage of the falling time of the droplets for the heating. The heating surface 111 can be the surface over or beneath which the heater is disposed. However, in some examples, the heating surface can correspond to a portion of the bottom surface or the side surface. The heating surface can cover 25% to 100% of the bottom surface or the side surface of the chamber 110. In some examples, the heating surface can cover 50% to 75% of the bottom surface or the side surface of the chamber 110. For example, FIG. 2D shows an example chamber 110 with a heating surface 111 covering a portion of the side surface, e.g. the top portion thereof. The heater 130 can be placed against this portion of the side surface. The bottom portion of the side surface may form a waste bottom 115 to gather the droplets. The waste bottom 115 is a volume of the chamber 110 where the fallen droplets accumulate while avoiding interference with the action carried out on droplets not yet in the waste bottom 115. The waste bottom 115 is a lowest portion of the chamber 110 below the heating surface 111.

The features of the example of FIG. 2C can be combined with the features of the example of FIG. 2A. The combination of the features of FIG. 2C and FIG. 2A can enable both to take advantage of the falling time of the droplets for the heating and to drive the droplets out of the chamber 110. Therefore, within the chamber 110, the PCR reactions can be carried out continuously. The features of the example of FIG. 2C can be combined with the features of the example of FIG. 2B. The combination of the features of FIG. 2C and FIG. 2A can enable both to separate droplets into groups and take advantage of the falling time of the droplets for the heating. The features of the example of FIG. 2C can be combined with the features of the example of FIG. 2A and the features of the example of FIG. 2B. This can enable for example a multiplexing continuous analysis.

The features of the example of FIG. 2D can be combined with the features of the example of FIG. 2A. The combination of the features of FIG. 2D and FIG. 2A can enable to heat the droplets while they are falling along the heating surface 111-, have a volume to accumulate the fallen droplets and a way to drive these fallen droplets out of the chamber 110 when it is desired to do so. The features of the example of FIG. 2D can be combined with the features of the example of FIG. 2B. The combination of the features of FIG. 2D and FIG. 2A can enable to separate droplets into groups, take advantage of the falling time of the droplets for the heating, and have a volume to accumulate the fallen droplets. The features of the example of FIG. 2D can be combined with the features of the example of FIG. 2A and the features of the example of FIG. 2B. This can enable for example a multiplexing analysis with accumulation of the fallen droplets at the bottom of the chamber 110 and a way to drive the droplets out of the chamber 110 for example when it is desired that the chamber 110 be emptied.

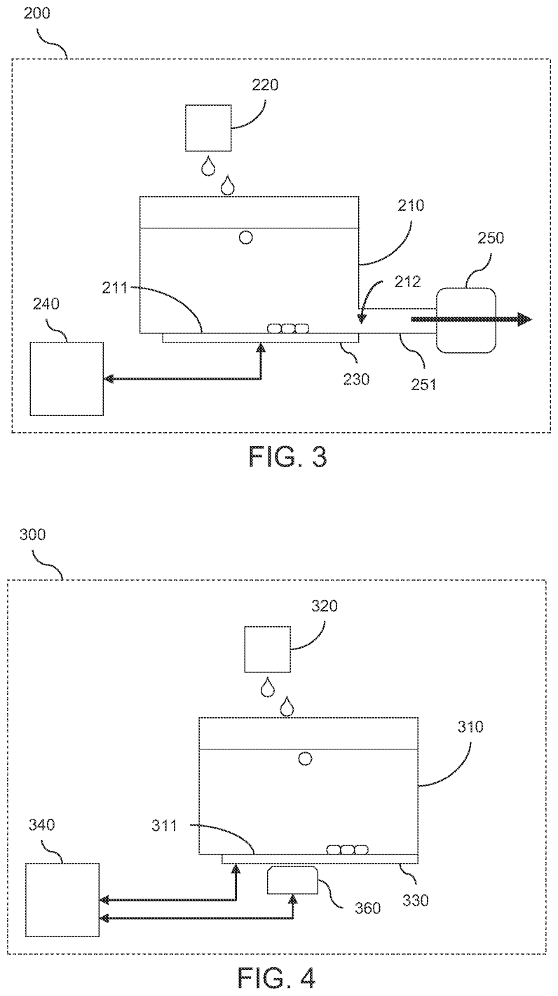

FIG. 3 is a schematic diagram showing another example droplet PCR system 200. This droplet PCR system 200 can comprise the same elements as the droplet PCR system 100 of FIG. 1—i.e. at least a chamber 210 with a heating surface 211, a droplet dispenser 220, a heater 230 and a controller 240. Droplet PCR system 200 can comprise a pump 250 and a connecting channel 251 connected with the chamber outlet 212 and the pump 250, wherein the pump 250 can drive the droplets out of the chamber through the connecting channel 251.

The pump 250 can be a peristaltic pump or an inertial pump such as a resistive microheater inside a micrometric channel. The resistive microheater can be one of the type used in thermal inkjet printing that vaporizes a thin layer of fluid to produce a drive bubble while the surrounding environment remains at a lower temperature, for example room temperature. The bubble pushes the fluid through a network of adjacent microchannels. The pump 250 can also be a laser-induced cavitation generator to create the bubbles.

Although not illustrated, the droplet PCR system 200 can comprise a waste tank to receive the droplets which are driven out of the chamber 210. The waste tank can be fluidly connected to the chamber 210 through the pump 250. The waste tank can also be placed at an elevation level lower than the outlet of the pump 250 and the droplets driven out of the chamber are poured or dispensed into the waste tank.

The droplet PCR system 200 can comprise a recycling tank to recycle the carrier fluid. The droplet PCR system 200 can comprise a separator to separate the droplets from the carrier fluid: the droplets being directed to the waste tank and the carrier fluid to the recycling tank. The droplet PCR system 200 can comprise a recirculation circuit to recirculate the carrier fluid inside the recycling tank back to the chamber 210. The separator can be a filter with apertures or pores sufficiently small to prevent the droplets to pass through while allowing the carrier fluid through. The separator can be a microfluidic particle separator with a channel network to separate the droplets from the carrier fluid. The waste tank and the recycling tank can form a single tank separated by the separator, the waste tank being upstream the recycling tank.

FIG. 4 is another example droplet PCR system 300. Droplet PCR system 300 can comprise the same elements as the droplet PCR system 100 of FIG. 1—i.e. at least a chamber 310 with a heating surface 311, a droplet dispenser 320, a heater 330 and a controller 340. Droplet PCR system 300 can comprise a detector 360 to detect signals from the droplets. The chamber 310 can be as described above. The detector 360 can be placed facing the heating surface 311 of the chamber 310 and the heater 330 to detect signal passing through: the heating surface 311 and the heater 330 being transparent to the signal or having a transmittance of the signal of at least 80%. The controller 340 can control the detector 360.

In some examples of heating surfaces 311 covering a portion of the side surface of the chamber 310 such as the case depicted for example in FIG. 2D, the detector can be placed facing an area of the side surface below the heating surface 311 to detect signals from falling droplets after they are pulse heated. A greater surface of the heating surface 311 can thus be used for the pulse heating necessary for the PCR reaction.

The detector 360 can in some examples be a fluorescence detector to detect fluorescence coming from the droplets, for example fluorescent emitted by detached fluorescent reporter dyes. In some examples, the detector 360 can be a luminescent detector to detect luminescence coming from the droplets, for example luminescence emitted by luminescent probes. In some examples, the detector 360 can be a radioactive detector to detect radioactive emission coming from the droplets, for example radioactive emission from radioactive probes. The detector 360 can in some examples be a point detector, i.e. detecting signals emitted by an area of the size of the droplet, or a line detector, i.e. detecting signals emitted by an area with a width of the size of the droplet and of various lengths. In such case, the detector 360 can be movable to scan the whole area of the heating surface 311. The detector 360 can cover the whole area of the heating surface 311. The detector 360 can be an array of sensors. The sensors can be a complementary metal oxide semiconductor (CMOS) sensors, a charge-couple device (CCD) sensors or an avalanche photodiode (APD) sensors. Thus, the array of sensors can be a CMOS array, a CCD array or an APD array.

The detector 360 can be immobile and the droplets can be caused to move towards the detector 360. The detector 360 can be movable to detect signals from droplets located at different locations.

The controller 340 can control the operation of the detector 360. An analyzer can be connected to the detector 360 by wire or wirelessly to analyze the detected signals. Analysis can comprise quantification of copies of labelled or marked DNA fragments, for example by a DNA intercalating dye or a fluorescent-labelled oligonucleotide probe.

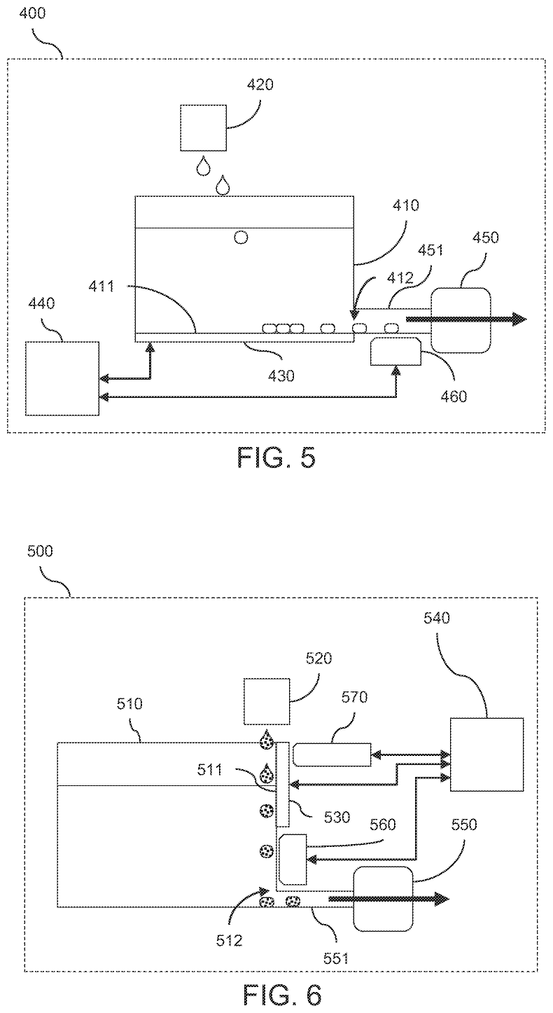

FIG. 5 is a schematic diagram of another example droplet PCR system 400. Droplet PCR system 400 can comprise the same elements as droplet PCR system 200 of FIG. 3—i.e. at least a chamber 410 with a heating surface 411 and an outlet 412, a droplet dispenser 420, a heater 430 and a controller 440, and a pump 450 fluidly connected to the chamber 410 through a connecting channel 451. Droplet PCR system 300 can comprise a detector 460. The detector 460 can be the same as the one described above with reference to FIG. 4. In the droplet PCR system of FIG. 5, the detector 460 can be placed facing the connecting channel 451 to detect signals from the droplets when the droplets are driven out of the chamber 410 through the connecting channel 451 and flowing pas the detector 460. In some examples where the chamber 410 comprises dividers, compartments, an outlet 412 for each compartments and a connecting channel 451 for each outlet 412, the detector 460 can span through all connecting channels 451 or the droplet PCR system 400 can comprise a plurality of detectors 460, so that one can be placed facing each connecting channels 451.

Droplet PCR system 400 can also comprise a waste tank, a recycling tank as described above, or both.

FIG. 6 is a schematic diagram of another example droplet PCR system 500. Droplet PCR system 500 can comprise the same elements as droplet PCR system 400 and illustrates the case where the chamber 510 is according to FIGS. 2A and 2D in combination—i.e. the chamber 510 can comprise an outlet 512 and the heating surface 511 can cover a top portion of the side surface of the chamber 510. Thus, droplet PCR system 500 can comprise at least the chamber 510, a droplet dispenser 520, a heater 530, a controller 540 and a pump 550 with a connecting channel 551 between the chamber 510 and the pump 550. The detector 560 can be placed facing the side surface below the heating surface 511.

Droplet PCR system 500 can comprise a confiner to confine the droplet to one part of the chamber 510. A confiner is understood herein as a component capable of confining the droplets to one part of the chamber 510, to limit the presence of the droplets within the one part of the chamber 510. This can make it possible to keep the droplets close to the heating surface 511, notably for the heating and detection.

In the example illustrate in FIG. 6, the confiner can be a magnet 570 placed facing the heating surface 511 and creating a magnetic field close to the heating surface 511 to attract droplets having magnetic particles therein to the heating surface 511. The magnetic particles are particles with magnetic properties: they can interact with the magnetic field created by the magnet 570 by being attracted towards the magnet 570. The magnetic particles can comprise iron, nickel, cobalt or a mixture thereof. The magnetic particles can be magnetic nanoparticles, for example with a diameter of 1 to 100 nm.

The magnet 570, when movable, can also be used to direct the droplets to desired locations within a plane parallel to the heating surface 511, wherein the movement of the magnet 570 can cause a change in the created magnetic field and eventually cause the magnetic particles to be attracted to the desired location and drag the droplet along with them.

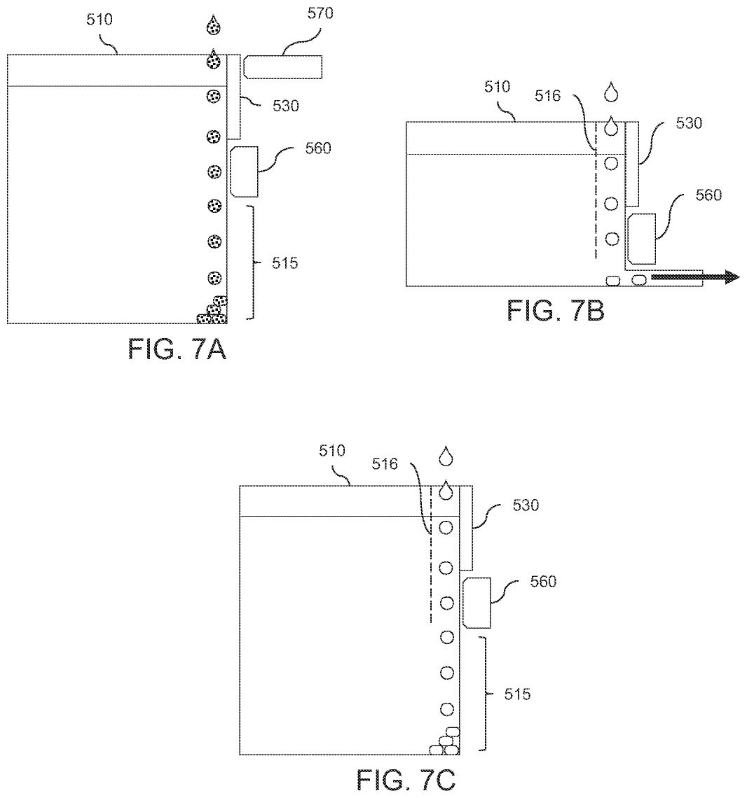

FIG. 7A is a schematic diagram focused on the chamber 510, the heater 530, the detector 560 and the magnet 570 of another example droplet PCR system. The chamber 510 can comprise a waste bottom 515 as in FIG. 2D to collect the droplets. The droplets can remain in the waste bottom 515. The droplets can be driven out of the chamber 510 when the chamber 510 comprises an outlet and is connected to a pump.

In FIGS. 7B and 7C, the confiner can be a mechanical confiner. A mechanical confiner is a structural element which limits the space where the droplets can move to. The droplets are thus confined because they cannot go through the mechanical confiner. The mechanical confiner can be a wall or membrane 516 within the chamber 510. The wall or membrane 516 is in this example parallel to the heating surface 511. The wall or membrane 516 has apertures or pores sufficiently small to prevent the droplets from passing through the wall or membrane 516 and sufficiently large to let the carrier fluid through. In some examples, the apertures or pores can have a diameter smaller than the diameter of the droplets, for example at least twice as small such as at most 5 μm. In some examples, the apertures or pores can have a diameter larger than 50 nm. The wall or membrane 516 can extend over the whole height of the chamber 511, i.e. they extend from the bottom surface up to the top of the chamber 511. The wall or membrane 516 can extend over a portion of the height of the chamber 511, for example a top portion. FIG. 7B illustrates the same configuration as in FIG. 6 except that the confiner is different. FIG. 7C illustrates the same configuration as in FIG. 7A except that the confiner is different. The confiner of FIGS. 7B and 7C can be used with droplets without magnetic particles, but droplets with magnetic particles can still be used with the confiner of FIGS. 7B and 7C for other desired purposes.

The outlet 512 can be positioned on the same surface of the chamber 510 as the heating surface 511 when this latter is a side surface of the chamber 510. The droplets then can fall down along the heating surface 5101 of the chamber 510 to the bottom surface thereof and in the vicinity of the outlet 512. The droplets can be easily driven out of the chamber 510.

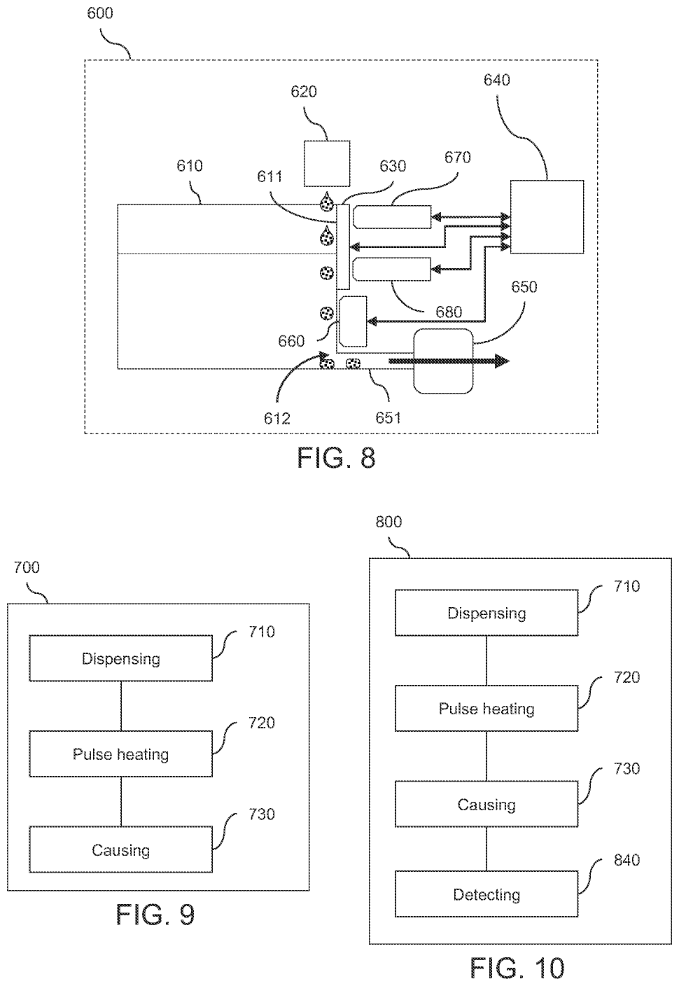

FIG. 8 is a schematic diagram of another example droplet PCR system 600. Droplet PCR system 600 can comprise a mixer 680 to mix the reagents within the droplets. A mixer 680 is an element which creates movements inside the droplets, for example by forcing some components inside the droplets to move, their movement in turn can create a turbulent flow within the droplets that mixes all the components together, thereby increasing contact of the PCR reagents with one another. Droplet PCR system 600 can comprise the same elements as droplet PCR system 500—i.e. at least a chamber 610 with a heating surface 611 and an outlet 612, a droplet dispenser 620, a heater 630, a controller 640, a pump 650 connected to the chamber 610 through a connecting channel 651, a detector 660, and a magnet 670 acting as a confiner. The mixer 680 can be placed facing the heating surface 611 to mix the reagents within the droplets moving in the vicinity of the heating surface 611 or resting thereon.

The mixer 680 can be a rotatable magnet to create a rotating magnetic field and set magnetic particles within the droplets in motion causing mixing of the reagents within the droplets. The rotating magnetic field can be created by a plurality of magnets driven by a gear train. The rotating magnetic field can be used to reduce or mitigate magnetic beads clumping. In FIG. 8, the magnet 680 acting as a mixer is illustrated as placed below the magnet 670 acting as a confiner: but their positions can be reversed. The magnetic 680 acting as a mixer can be used without the magnetic 670 acting as a confiner. Both the magnetic 680 acting as a mixer and the magnetic 670 acting as a confiner can act as a confiner and as a mixer at the same time.

Therefore, in some examples, the droplet PCR system can comprise a magnet to: drive droplets containing magnetic particles to a desired location (FIGS. 6, 7A, 7B, 7C and 8); or generate a rotating magnetic field to mix reagents inside particles containing magnetic particles (FIG. 8).

Although not illustrated, the droplet PCR system 100, 200, 300, 400, 500, 600 can comprise a heat sink in contact with a surface of the chamber to dissipate heat accumulated by the carrier fluid. A heat sink is a passive heat exchanger which transfers the heat of the carrier fluid to the environment outside the droplet PCR system. The heat sink can comprise a base and a plurality of fins extending from the base and placed in parallel to one another. The base is in contact with a surface of the chamber 110, 210, 310, 410, 510, 610 away from the heating surface 111, 211, 311, 411, 511, 611. Heat from the droplets transfers to the carrier fluid. Heat from the carrier fluid transfers to the surface of the chamber in contact with the base of the heat sink.

At least part of the droplet PCR system can be formed in a microfluidic chip. A microfluidic chip is a device which integrates one or a plurality of elements of the droplet PCR system on a single integrated circuit. The size of the integrated circuit can be 25 mm³ to 25 cm³ although smaller and larger integrated circuits are also possible. The elements which can be formed on the microfluidic chip are: the chamber, the divider, the separator, the connecting channels, the pump, the waste tank and the recycling tank.

Any component as described more in particular with reference to one of the figures, can be used in the system illustrated by the other figures.

Some examples relate to a droplet PCR method comprising: dispensing PCR-reagent-containing droplets into a chamber filled with a carrier fluid, wherein the chamber acts as a heat sink: pulse heating a fluid layer adjacent one surface of the chamber while maintaining the rest of the chamber at a lower temperature: and causing the dispensed droplets to fall along the one surface or rest thereon.

FIG. 9 is a block diagram of an example droplet PCR method 700 of such a droplet PCR method. In block 710 PCR-reagent-containing droplets are dispensed into a chamber filled with a carrier fluid immiscible with the droplets or hardly miscible with the droplets, causing in block 730 the dispensed droplets to fall along a heating surface of the chamber or rest thereon. In block 720 a layer adjacent the heating surface is pulse heated while maintaining the rest of the chamber at a lower temperature.

Dispensing PCR-reagent-containing droplets can comprise dispensing aqueous droplets comprising PCR-reagents and wherein the carrier fluid can be an oil.

Dispensing PCR-reagent-containing droplets can comprise dispensing droplets with a diameter which is at least 10 times smaller than a dimension of the chamber taken perpendicularly to the heated surface of the chamber. Thus, in block 710 droplets can be dispensed with a diameter at least 10 times smaller than a dimension of the chamber perpendicular to the heating surface, for example dispensing droplets with a diameter at least 50 times, at least 100 times, at least 200 times, at least 500 times, or at least 1000 times smaller than a dimension of the chamber perpendicular to the heating surface.

In block 710 droplets can be dispensed at one location above the chamber or at different locations above the chamber.

In block 710, DNA, DNA polymerase, nucleotides and a primer set can be mixed to form a droplet and the droplet is then ejected. DNA can be provided alone or in a mixture with DNA polymerase, nucleotides or both.

In block 710, RNA, reverse transcriptase, DNA polymerase, nucleotides and a primer set can be mixed to form a droplet and the droplet is then ejected. RNA can be provided alone or in a mixture with reverse transcriptase, DNA polymerase, nucleotides or a combination thereof.

The primer set can be provided as a mixture of primers or formed on demand from a plurality of primer reservoirs. Block 710 can be carried out in rounds. Each round can comprise dispensing a plurality of droplets. Each round can correspond to a different primer set.

When the dispensed droplets are caused to fall along the heating surface, the droplets can be pulse heated during their fall, i.e. the droplets can fall within the heated fluid layer adjacent the heating surface. When the dispensed droplets are cause to rest on the heating surface, the droplets can be pulse heated after they land on the heating surface, i.e. they can be heated once they reach the area of the heated fluid layer adjacent the heating surface. Thus, pulse heating can comprise heating the droplets while the droplets are moving or resting.

In some examples, in block 720 the DNA polymerase can be activated by pulse heating the layer at a temperature of 75° C., to 100° C., for example 75° C., to 80° ° C., 94° C., to 96° ° C. or 98° C., to initialize the PCR reaction. The activation of the DNA polymerase is used when the DNA polymerase is a DNA polymerase which is capable of efficiently replicating DNA within a temperature range, and when outside of the temperature range, its activity is reduced if not absent. Taq polymerase, originally isolated from a thermophilic eubacterial microorganism Thermus aquaticus, is an example of such DNA polymerase.

In some example, in block 720, the DNA can be denatured by pulse heating the layer at a temperature of 94° C., to 98° C. During denaturation, hydrogen bonds between complementary bases within DNA responsible for the double-strand structure of DNA are broken yielding two single-stranded DNA.

In some examples, in block 720 the primers can be annealed to the denatured DNA by pulse heating the layer to cause the primers to hybridize with denatured DNA. During annealing, the primers hybridize with complementary sites within single-stranded DNA if such sites are present. The choice of temperature affects the strength of the bonding between the primers and the single-stranded DNA. Thus, the temperature to which the fluid layer is heated can be 3 to 5° C. below the melting temperature of the primers used. In some examples, the temperature to which the fluid layer is heated can be 50° ° C., to 60° C.

In some examples, in block 720, the primers can be elongated by pulse heating the layer at a temperature of 70° ° C., to 85° C., for example 72° C., or 75° ° C., to 80° ° C., to cause the DNA polymerase to synthesize new DNA strands from the primers and the nucleotides. During elongation, the DNA polymerase synthesizes a new DNA strand complementary to the single-stranded DNA to which the primers have hybridized by adding free nucleotides to the primers.

Denaturation, annealing and elongation together constitute a single PCR cycle. In some examples, at each cycle, the number of copies of the DNA segments targeted by the primers is doubled. Therefore, after n cycles, the number of copies of each target DNA segment is 2″ if the target DNA segment is initially present in the droplet, where n is an integer different from 0.

In some examples, at each cycle: the number of copies of the DNA segments targeted by the primers is multiplied by k, with 1≤k≤2. Therefore, after n cycles, the number of copies of each target DNA segment is kⁿ if the target DNA segment is initially present in the droplet, where n is an integer different from 0.

In some example, in block 720 the fluid layer is pulse heated at a temperature of 70° C., to 74ºC for a final elongation after all desired PCR cycles are completed. During the final elongation, any incomplete elongation of copies of single-stranded DNA is completed.

The total heating cycle can be few microseconds to few seconds, for example from 1 μs to 10 s or 10 μs to 10 s.

In some examples, in block 720, RNA can be reversed transcribed into a complementary DNA. During the reverse transcription, the reverse transcriptase creates double-stranded DNA from RNA and nucleotides.

Block 720 can be carried out after each round of dispensing 710, after a given number of rounds of dispensing 710 or after all rounds of dispensing 710 are completed.

FIG. 10 is a block diagram of an example droplet PCR method 800. Example droplet PCR method 800 can comprise the blocks of droplet PCR method 700. In this example, the droplet PCR method can comprise detecting light signals emitted by the droplets while the droplets are moving or resting. Thus, in block 840 light signals from the droplets can be detected while the droplets are moving or resting. In some example, in block 840 the light signals can be detected while the droplets are falling to the bottom of the chamber or when they are driven out of the chamber. In some example, in block 840, signals from one droplet can be detected at a time. In some example in block 840, signals from a plurality of droplets can be detected at a time. In some example, in block 840, the signals can be detected during the PCR reaction caused by pulse heating of block 720 or after the PCR reaction is completed. In some example, in block 840, the signals can be detected separately after each round of dispensing of block 710 is completed. In some example, in block 840, the signals can be detected after a given number of rounds of dispensing of block 710 are completed. In some example, in block 840, the signals can be detected after all rounds of dispensing of block 710 are completed.

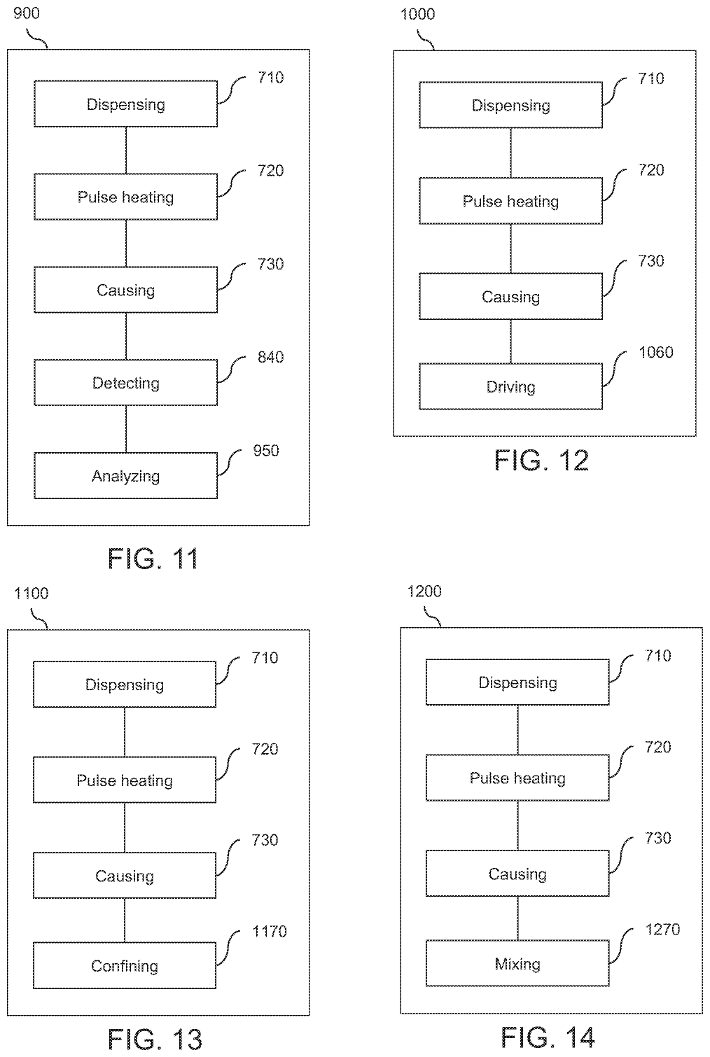

FIG. 11 is a block diagram of an example droplet PCR method 900. Example droplet PCR method 900 can comprise the blocks of droplet PCR method 800 and block 950. In block 950 the detected signals can be analyzed. In some example, in block 950, the detected signals can be analyzed after all rounds of detecting are completed. In some example, in block 950, the detected signals can be analyzed after each round of detecting is completed. In the latter case. Droplet PCR method 800 can comprise controlling the next round of droplets dispensing according to the results of the signal analysis. Controlling the next round of droplets dispensing can comprise selecting a primer set to be dispensed with the sample preparation. For example, after each round of dispensing of block 710, heating of block 720, causing of block 730 and detecting of block 840, the detected signals can be analyzed and the result of the analysis can be used to control the next round of dispensing of block 710, heating of block 720, causing of block 730 and detecting of block 840: at each new round, the primer set can be changed. In some example, in block 950, analysis can comprise quantitatively measuring copies of target DNA segments which can be labelled or marked.

FIG. 12 is a block diagram of an example droplet PCR method 1000 comprising the blocks of droplet PCR method 700 and block 1060. Droplet PCR method 1000 can comprise driving the droplets: to a desired location within the chamber while the droplets are falling; or out of the chamber. Thus, in block 1060, the droplets can be driven to desired locations within the chamber while the droplets are falling; or out of the chamber. In some examples, in block 1060, a magnetic field can be generated to attract magnetic particle containing droplets and moved to drive the droplets in the desired locations.

FIG. 13 is a block diagram of an example droplet PCR method 1100. Example droplet PCR method 1100 can comprise the blocks of droplet PCR method 700 and block 1170. In block 1170, the droplets can be confined within a given zone of the chamber. In some example, in block 1170, a magnetic confinement or a mechanical confinement can be used. For example, magnetically confining can comprise generating a magnetic field to attract magnetic particle containing droplets to keep them close to the heating surface. Mechanically confinement can involve the use of a wall or a membrane with apertures or pores preventing the droplets from passing through while allowing the carrier fluid to pass through.

FIG. 14 is a block diagram of an example droplet PCR method 1200. Example droplet PCR method 1200 can comprise the blocks of droplet PCR method 700 and block 1270. Droplet PCR method 1200 can comprise remotely mixing the reagents inside the droplets. Thus, in block 1270, the reagents inside the droplets can be remotely mixed. In some example, in block 1270, a rotating magnetic field can be generated inside the chamber to mix the reagents inside the droplets. In some example, in block 1270, the reagents inside falling droplets or resting droplets can be mixed.

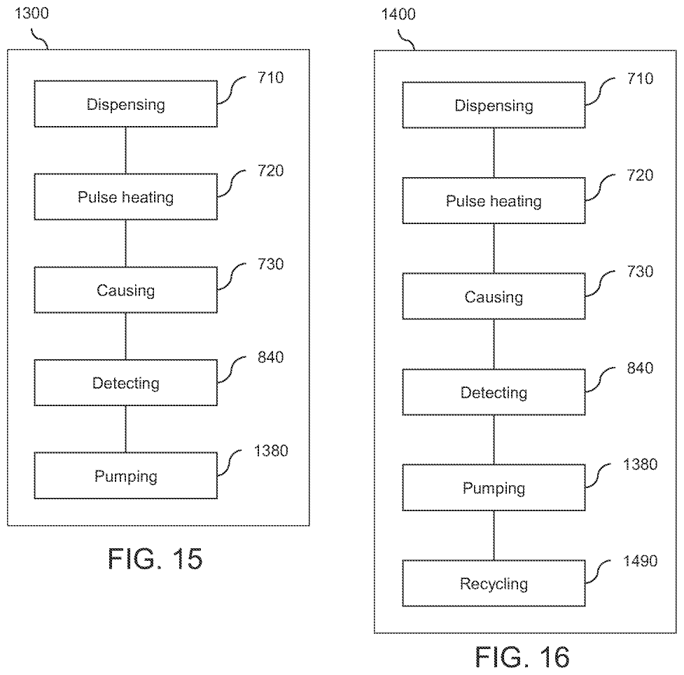

FIG. 15 is a block diagram of an example droplet PCR method 1300. Example droplet PCR method 1300 can comprise the blocks of droplet PCR method 800 and block 1380. In block 1380, the droplets can be pumped out of the chamber. In some example, in block 1380, the droplets can be pumped out of the chamber after each round of detection of block 840 is completed. In some example, in block 1380, the droplets can be pumped out of the chamber after all rounds of detection of block 840 are completed. In some example, in block 1380, the droplets can be pumped out of the chamber at the same time detection of block 840 is carried out for a continuous method of analysis. In this latter case, the droplets can be pumped out of the chamber and detection is carried out as the droplets flow past the detector.

FIG. 16 is a block diagram of an example droplet PCR method 1400. Example droplet PCR method 1400 can comprise the blocks of droplet PCR method 1300 and block 1490. In block 1490 the carrier fluid can be recycled. In some example, in block 1490, the droplets can be separated from the carrier fluid. The carrier fluid can be collected and injected back into the chamber.

Any block as described more in particular with reference to one of the figures, can be used in the method illustrated by the other figures.

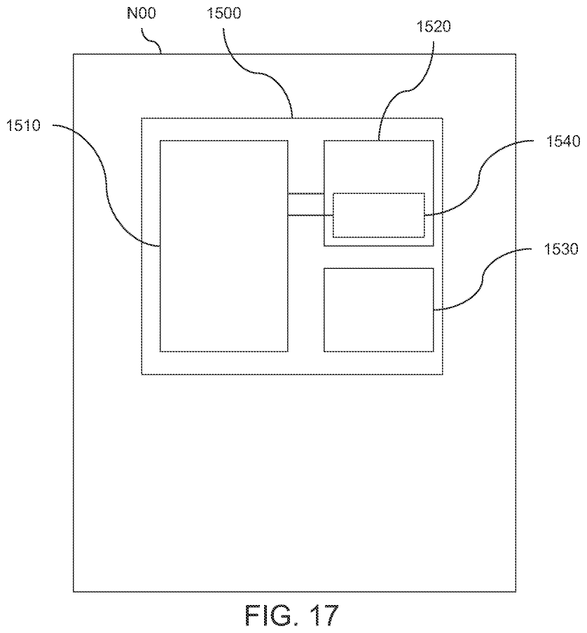

FIG. 17 is a schematic diagram of a computer system 1500. Computer system 1500 can be used to control any of the droplet PCR systems N00 described above, N being 1-6. The computer system 1500 can be the controller 140, 240, 340, 440, 540, 640 for the droplet PCR systems herein described. The computer system 1500 comprises a processor 1510, a storage 1520 coupled to the processor 1510, an interface 1530 to a droplet PCR system with a droplets dispenser and a heater, and an instruction set 1540 to cooperate with the processor 1510 and the storage 1520 to control the droplet PCR dispenser to dispense PCR-reagent containing droplets, and control the heater to produce a pulsed heat flux.

The processor 1510 can be a digital circuit capable of performing operations on digital data. The processor 1510 can be a central processing unit (CPU), a multi-core processor or a front-end processor.

The storage 1520 can be any electronic, magnetic, optical, or other physical storage device that stores executable instructions. The storage 1520 can be a Random Access Memory (RAM) storage, an Electrically-Erasable Programmable Read-Only Memory (EEPROM), a storage drive, an optical disc, and the like. In some example implementations, the storage can be a machine-readable medium which may be a tangible, non-transitory medium, where the term “non-transitory” does not encompass transitory propagating signals. The storage may be disposed within a processor-based system such as computer system 1500, in which case the executable instructions may be deemed “installed” on the processor-based system. Alternatively, the storage may be a portable (e.g., external) storage medium, for example, that allows a processor-based system to remotely execute the instructions or download the instructions from the storage medium. In this case, the executable instructions may be portion of an “installation package”. The storage may be encoded with a set of executable instructions such as set 1540.

The interface 1530 can be an interface to components of the droplet PCR system. The interface 1530 can be an electrical connector between the processor 1510 and the components of the droplet PCR system.

The instruction set 1540 can comprise instructions to perform a droplet PCR method using a droplet PCR system according to any of the examples hereby described.

1. A droplet PCR system comprising:
a chamber having a heating surface;
a droplet dispenser to dispense PCR-reagent-containing droplets into the chamber;
a heater disposed onto the heating surface of the chamber to heat a layer of fluid adjacent the heating surface; and
a controller to control the heater to produce a pulsed heat flux.

2. The droplet PCR system of claim 1, wherein the chamber is filled with a carrier fluid.

3. The droplet PCR system of claim 2, wherein the droplet dispenser dispenses aqueous droplets and the carrier fluid is an oil.

4. The droplet PCR system of claim 1, wherein the chamber has a dimension taken perpendicularly to a portion of the heating surface of at least 50 μm.

5. The droplet PCR system of claim 1, wherein the chamber comprises an outlet to discharge the droplets out of the chamber.

6. The droplet PCR system of claim 5, comprising a pump and a connecting channel fluidly connected with the chamber outlet and the pump, wherein the pump drives the droplets out of the chamber through the connecting channel.

7. The droplet PCR system of claim 1, wherein the heating surface is a portion or the entirety of a bottom surface of the chamber.

8. The droplet PCR system of claim 1, wherein the heating surface is a portion or the entirety of a side surface of the chamber.

9. The droplet PCR system of claim 1, comprising a detector to detect signals from the droplets.

10. A droplet PCR method comprising:
dispensing PCR-reagent-containing droplets into a chamber filled with a carrier fluid, wherein the chamber acts as a heat sink;
pulse heating a fluid layer adjacent one surface of the chamber while maintaining the rest of the chamber at a lower temperature; and
causing the dispensed droplets to fall along the one surface or rest thereon.

11. The droplet PCR method of claim 10, wherein dispensing PCR-reagent-containing droplets comprises dispensing aqueous droplets comprising PCR-reagents and wherein the carrier fluid is an oil.

12. The droplet PCR method of claim 10, wherein dispensing PCR-reagent-containing droplets comprises dispensing droplets with a diameter which is at least 10 times smaller than a dimension of the chamber taken perpendicularly to the heated surface of the chamber.

13. The droplet PCR method of claim 10, wherein pulse heating comprises heating the droplets while the droplets are moving or resting.

14. The droplet PCR method of claim 10, comprising detecting light signals emitted by the droplets while the droplets are moving or resting.

15. The droplet PCR method of claim 10, comprising driving the droplets:
to a desired location within the chamber while the droplets are falling; or out of the chamber.
